(19) United States
(12) Patent Application Publication
(10) Pub. No.: US 2004/0265855 A1
(43) Pub. Date: Dec. 30, 2004

(54) High-throughput DNA-isolation and transfection for analysing the function of genes or genetic products

(75) Inventors: Ulrich Pessara, Weilheim (DE); Michael Kazinski, Munchen (DE); Johannes Gorl, Munchen (DE)

Correspondence Address: ROPES & GRAY LLP, ONE INTERNATIONAL PLACE, BOSTON, MA, 02110-2624, US

(73) Assignee: Xantos Biomedicine AG, Munchen (DE)

(21) Appl. No.: 10/773,100

(22) Filed: Feb. 5, 2004

Related U.S. Application Data
(63) Continuation-in-part of application No. PCT/EP02/08962, filed on Aug. 9, 2002.

(30) Foreign Application Priority Data
Aug. 10, 2001 (EP) 01119347.1

(51) Int. Cl. C12Q 1/68

(52) U.S. Cl. 435/6

(57) ABSTRACT

The present invention relates to a method for screening a collection of nucleic acid molecules for a desired property of the nucleic acid or of a (poly)peptide encoded thereof, comprising the steps (a) automated picking of the cell collection containing the collection of nucleic acid molecules with a first robot; (b) automated lysis of the cells with a second robot; (c) automated separation of the cell DNA from the cell debris with a second robot; (d) optionally automated separation of endotoxins from the DNA with the second robot if the cells are bacteria; (e) automated transfection of the cells with the DNA obtained in step (c) or, if the cells are bacteria, with the DNA obtained in step (d) with a third robot; and (f) automated screening for the desired property with a fourth robot. Moreover, the invention relates to methods for the enhancement of the binding properties of the (poly)peptide identified by the of the screening method of the invention or encoded by the DNA identified and isolated and a method for the production of a pharmaceutical composition on the basis of (poly)peptides which can be obtained with the method of the invention and moreover the formulation of the substance obtained with a pharmaceutically acceptable carrier or dilutent.

The present invention relates to a method for screening a collection of nucleic acid molecules for a desired property of the nucleic acid or a (poly)peptide encoded thereby, the method comprising the steps (a) automated picking of a collection of cells containing the collection of nucleic acid molecules by means of a first robot; (b) automated lysis of the cells by means of a second robot; (c) automated separation of the cell DNA from cell debris by means of the second robot; (d) optionally automated separation of endotoxins from the DNA by means of the second robot if the cells are bacteria; (e) automated transfection of cells with the DNA obtained in step (c) or, if the cells are bacteria, with the DNA obtained in step (d) by means of a third robot; and (f) automated screening for the desired property by means of a forth robot. Moreover, the invention relates to methods for improving the binding properties of the (poly)peptide which is identified by the screening method of the invention or encoded by the infected or isolated DNA, as well as to methods for producing a pharmaceutical composition on the basis of (poly)peptides obtainable by the method of the invention and, furthermore, to the formulation of the substance obtained with a pharmaceutically acceptable carrier or diluent.

In the specification, a number of prior art documents is cited. The disclosure content of these documents is herewith incorporated by reference in its entirety in the present description.

For years, high through-put screening has been a tried and tested instrument for finding potential active agents in pharmaceutical research. It is, however, relatively new to use said high through-put technology also for methods such as the isolation of DNA from bacteria and the transfection of cellular systems. In particular, the screening of cDNA libraries is of interest in this case. The screening of cDNA or generic libraries which are usually cloned in bacteria requires a process that can generally be divided into four steps and comprises 1) the picking of the bacteria colonies, 2) the preparation of DNA, 3) the transfection of DNA and 4) the reading out of a functional test.

The DNA is usually isolated from bacteria by means of two different methods: alkaline lysis of bacteria with subsequent purification of the DNA recovered over columns or adhesion of the DNA obtained by the alkaline lysis to special micro-particles (so-called beads).

A protocol for alkaline lysis has, for instance, been described in Sambrook et al., “Molecular Cloning, A Laboratory Handbook”, CSH Press, Cold Spring Harbor 1989; or Ausubel et al.; Current Protocols, in Molecular Biology 2002; John Wiley & Sons, Inc., N.Y. Methods for purifying DNA, RNA or their hybrids with magnetic silica beads have been described for instance in U.S. Pat. No. 6,027,945 or WO 98/31840. Removing cell debris by using magnetic micro-particles has been shown in U.S. Pat. No. 5,646,283.

Said purification is usually based on chemical purification methods and is therefore suitable to a very restricted extent for screening complex libraries.

Corresponding methods are designed to be used for carrying them out in a laboratory or on pipetting robots for a small through-put of samples. The daily through-put rate varies and, depending on the method, is limited to a maximum of 3000 to 6000 preparations per day. Due to this limited through-put rate of samples, this method is not suitable for high through-put.

For transfecting DNA in eukaryotic cell systems, chemical methods such as lipofection fulfil the requirements for a high through-put rate of samples. The DNA can be introduced into the cell by the preparation of cell membrane-permeable DNA complexes or by penetration or fusion with the cell membrane. Physical methods such as magnetofection or electroporation, too, are suitable methods for high through-put.

Single steps of screening processes of complex libraries can be carried out in an automated manner already. Corresponding devices can be purchased from Beckman Coulter or Tecan. The devices Biomek 2000 (Beckman Coulter; Fullerton, USA) or Genesis (Tecan; Durham, USA) are semi-automated working platforms for the use of microtitre plates. These systems are general working platforms which can, for instance be adapted to the use for DNA preparation. The possibilities of application, however, are limited as, for example, no centrifuges are integrated. Thus, advantageous test protocols such as, for instance, preparing a DNA by alkaline lysis (mini-prep) cannot be carried out. Moreover, manual steps such as, e.g., for pelleting/precipiting the bacteria are not necessary.

An automated high through-put DNA preparation system for the use of microtitre plates has been described in EP 569 115 A2. By integrating modified centrifuges, a DNA preparation after alkaline lysis is made possible. In so far, compared to the state-of-the-art processes described above, this method is already an improvement. However, a degree of purity of the DNA, which is required for the application of transfections, is not achieved. This is, amongst others, due to the fact that the DNA is still contaminated by endotoxins. It is also disadvantageous that this system, just like the Genesis (Tecan) and the Biomek 2000 (Beckman) systems are not outlined as conveyor road system or can be enlarged as such. It is therefore not possible to interconnect the individual process steps. The sample through-put rate of the aforementioned systems is thus limited to about 3000 to 6000 preparations/day at maximum.

PCT/EP00/00683 describes a method for the identification of nucleic acid sequences that do not have a selectable activity. The method comprises the steps of providing the DNA library, cultivating the host cells, preparing the DNA, transfecting the target cells with the target DNA and functional determination of the activity of the DNA in the target cell. This application is a method which has a certain degree of automation of the DNA preparation. Accordingly, embodiments of two robots which can each perform the DNA preparation and the DNA transfection are presented. With these methods, too, sample through-put rates in the range of more than 10³ preparations per day can be achieved.

PCT/EP00/13132 describes a screening method for nucleic acids which also includes nucleic acids with selectable activity. Apart from the screening method, also the automation of the method and a preferred embodiment for carrying out the DNA preparation and DNA transfection using single robots are recorded. With these methods, too, sample through-put rates in the range mentioned above can be achieved.

All aforementioned methods have the disadvantage that they are not suitable for screening complete gene libraries for molecules having the desired properties in a shorter period of time. For screening gene libraries that have, for instance, a complexity of up to or even more than 10⁶ cDNAs requires a high sample through-put rate per day in order to be easy to handle and to lead to the desired properties in a clear time frame. Such a sample through-put rate is not only made possible by optimising the individual processes described in the state of the art. It is rather necessary to try new ways, i.e. new combinations of processes have to be found, to subject gene libraries having a high degree of complexity to functional studies in an acceptable time frame that is appropriate for therapeutic developments. The technical problem underlying the present invention was to provide a method that meets these requirements.

This technical problem is solved by the embodiments characterised in the claims.

Accordingly, the invention relates to methods for screening a collection of nucleic acid molecules for a desired property of the nucleic acid or of a (poly)peptide encoded thereby, comprising the steps of (a) automated picking of a collection of cells containing the collection of nucleic acid molecules by means of a first robot; (b) automated lysis of the cells by means of a second robot; (c) automated separation of the cellular DNA from the cell debris by means of the second robot; (d) optionally automated separation of endotoxins from the DNA by means of the second robot if the cells are bacteria; (e) automated transfection of cells with the DNA obtained in step (c) or, if the cells are bacteria, obtained in step (d) by means of a third robot; and (f) automated screening for the desired property by means of a fourth robot.

Step (d) of the method of the invention is an optional step. Especially if the sensitivity of the preferably eukaryotic cells to be transfected to endotoxin is very low, this step is preferred, it can, however, also be left out.

Accordingly, the method of the invention either comprises steps (a), (b), (c), (d), (e) and (f) or the steps (a), (b), (c), (e) and (f).

According to the invention, the latter order of steps can also be defined as (a), (b), (c), (d′) and (e′), with step (d′) corresponding to step (e) and step (e′) corresponding to step (f).

Within the meaning of the invention, the term “collection” relates to a number of nucleic acid molecules which is more than 10³ different molecules, preferably at least more than 10⁴ different molecules, more preferably at least more than 10⁵ different molecules and most preferably 10⁶ different molecules such as 2×10⁶ or 3×10⁶ different molecules.

The “nucleic acid molecules” are preferably coding regions together with homologous or heterologous expression control sequences. It is particularly preferred that they represent or substantially represent the genome of an organism.

Said organism can be a prokaryote, e.g. a bacterium, or a eukaryote, e.g. a yeast. If the organism is a eukaryote, it is, in a preferred embodiment, a mammal, e.g. a human.

The term “(poly)peptide” describes both peptides and polypeptides (proteins).

According to the convention, a chain of up to 30 amino acids is called a peptide and a chain of more than 30 amino acids is called a polypeptide.

Within the meaning of the invention, the term “automated” means that the step in question is not performed by humans but is only carried by a machine. However, this definition of said terms, of course, also includes manipulations and adjustments of the machine (the robot) by humans.

Within the meaning of this invention, the term “cell debris” means the mass of cell components obtained after lysis of a cell and that can be separated from the aqueous, DNA-containing supernatant by centrifugation, e.g. at 3000×g. Cell debris usually contains proteins and, in the case of bacteria, cell membrane components.

The expression “robot” refers to an automated working station with grip arms and specific product processing stations such as, e.g. centrifuges, incubation places, etc.

With the method of the invention, a screening method is provided in which the four process steps of picking of the colonies, preparing of the DNA, transfecting of the DNA and reading out a functional screening assay are carried out in an automated manner by a robot. In this way, an automated overall process is made possible which is suitable for high through-put screening. The automated removal of endotoxins, preferably using magnetic micro-particles, can be considered an essential component of this method in one embodiment (i.e. an embodiment including step (d)). Only in this way, in combination with further automated steps, is an acceptable time frame for high through-put screening of libraries having a high degree of complexity achieved. For the purification of the DNA from endotoxins in this embodiment is an essential prerequisite for being able to use the DNA directly for the transfection. Only in this way can thus the DNA obtained from the DNA preparation be directly used for analyses and transfections. It is of particular advantage that the time-consuming centrifugation steps are considerably reduced. Methods for removing endotoxins from DNA, RNA or their hybrids using magnetic silica particles are described in U.S. Pat. No. 6,194,562 or WO 99/54340.

In another embodiment of the method of the invention (i.e. the embodiment without step (d)), the removal of the endotoxins is not essential. This is particularly the case if the cells to be transfected have a low sensitivity to endotoxins and are thus not essentially interfered with or killed by endotoxin contamination in common DNA purification processes.

The combination of the automated individual processes which are carried out by interconnected robots makes it, for the first time, possible to achieve a sample through-put rate of up to 30,000/40,000 samples per day. In other words, the combination of a serial production technique using the components described (according to the two above-described embodiments) makes it possible to achieve a through-put rate in the preparation of DNA capable for transfection which has never been achieved before. Using the same high through-put method, this DNA can be analysed for its biological function after transfection, preferable in eurkaryotic cells, which makes it possible to screen a complete cDNA gene library within one month.

In a preferred embodiment of the method of the invention, the collection of nucleic acid molecules is a gene library.

The term “gene library” is known in the state of the art and defined as a “Collection of cloned DNA fragments representing an entire genome” in Winnacker, “Gene und Klone”, VCH Weinheim 1985 (p. 403). The invention also includes gene libraries with gaps, i.e. which do not represent the entire gene or which represent an expression stage, e.g. of a certain tissue, a stage of a disease or a development.

In another preferred embodiment of the method of the invention, the nucleic acid molecules are genomic DNA or cDNA molecules or RNAi oligonucleotides. Corresponding RNAi oligonucleotides are synthesised for instance by Dharamcon (LaFayette, USA), Xeragon (Germantown, USA) or Ambion (Austin, USA).

In a particularly preferred embodiment of the method of the invention, the gene library is an expression cDNA gene library, preferably a eukaryotic gene library, a human gene library is particularly preferred.

The term “expression cDNA gene library”, too, is well-known in the state of the art. In an expression cDNA gene library, the cDNA molecules are cloned into an expression vector which allows their expression in a suitable host; cf. Winnacker, loc. cit. or Sambrook et al., “Molecular Cloning, A Laboratory Manual”; CSH Press, Cold Spring Harbour 1989.

The gene library is preferred to be normalised (i.e. the number of the genes contained in the gene library is virtually the same) and/or enriched for “full length cDNA”.

In another preferred embodiment, the collection of nucleic acids is a collection of clones. A collection of clones is a collection of selected cDNA clones which preferably has “full length cDNA”.

In a preferred embodiment of the method of the invention, the cells in step (a) and/or step (e) are mammalian cells, insect cells, yeast cells or bacteria.

Examples of mammalian cells are COS cells, HUVEC cells, Aspergillus(niger/nidulans etc.) cells or CHO cells. Examples of insect cells are Spodoptera frugiperda cells. Suitable yeast cells include cells of the species S. cerevisiae or P. pastoris. Suitable bacteria can be both Gram-negative and Gram-positive bacteria.

In a particularly preferred embodiment of the method of the invention, the bacteria are Gram-negative bacteria.

The particularly preferred properties of the method of the invention, are in particular of importance if the bacteria are Gram-negative bacteria as they, in particular, have endotoxins as cell wall or cell membrane components. With the Gram-negative bacteria, in particular bacteria of the species E. coli are used for cloning purposes in the state of the art.

In a most preferred embodiment of the method of the invention, the Gram-negative bacteria thus belong to the species E. coli.

It is particularly preferred that they are E. coli DH5α, E. coli Shure and E. coli JM 109.

In a preferred embodiment of the invention, at least one of the steps (a) to (f) (with or without step (d)) is carried out in microtitre plates.

Conventional microtitre plates have the advantage that, independent from the number of wells, they have a standardised size which renders them particularly suitable for an automated use by the robots. Microtitre plates (e.g. obtainable from Nunc), are usually made of PVC or polystyrene. They can have 6, 24, 96, 384 or 1536 wells. The microtitre plates that are preferably used in the method of the invention have 96 or 384 wells.

In a particularly preferred embodiment of the method of the invention, all steps (a) to (f) (with our without step (d)) are carried out in microtitre plates.

In an additional preferred embodiment of the method of the invention, the microtitre plates are marked with bar codes.

Therefore, this embodiment is particularly advantageous as it allows a complete tracking of all plates, also after changing from one robot to another. Thus, an assignment starting from plating the cells for processing by the first robot to functional screening and reading-out by the forth robot is particularly easy and can be done in a time-saving manner. In this was, it is easily possible to go back to the initial clones on the screening plate after the functional screening.

The bar code technique on the robots 2 and 3 makes it moreover possible that the individual processes are interlaced within the conveyor road system.

In another preferred embodiment of the method of the invention, the first robot is characterised by at least one and preferably all of the following features: (a) a digital image processing system for collecting the plated bacteria, (b) a working station with a grip arm for microtitre plates for transferring the microtitre plates between the processing stations, (c) a separation module having one or more heads with needles for picking the plated single colonies and for placing them into the microtitre plates, (d) integrated product processing stations for cleaning the needles between the working steps and replicating the placed single colonies in the microtitre plates and (e) a computer-based bar code identification and tracking system.

The microtitre plates are preferably plates with 96 or 384 wells. The integrated product processing stations include a sterilisation system. Moreover, it is preferred that the grip arm is a robot arm which has at least two heads with needles, wherein the heads are used for cross-picking and are cleaned on the sterilisation station. In addition, a modular set-up of the robot arm is preferred which allows an exchange of grip arm modules for separation head modules.

In a preferred embodiment of the method of the invention, the lysis is an alkaline lysis.

The conduction of the alkaline lysis is described, amongst others, in Sambrook, loc. cit., and in another passage of this description.

In an additional preferred embodiment of the method of the invention, the second robot is characterised by at least one and preferably all of the following features: (a) a conveyor road transport system combined with grip arms for the microtitre plates for reloading the products and for transferring the microtitre plates between the product processing stations, (b) product processing stations integrated into the transport system, particularly centrifuges, pipetting automats, shakers and incubation places for incubation at different temperatures, (c) a sensor technology for the detection of product positions as well as for the detection of errors, (d) a software for the interlaced handling of several processes which are in the machine for a continual production process and (e) a computer-based bar code identification and tracking system, preferably with an internal product tracking containing a time stamp function for the interlacing of time-critical sub-processes.

In this case, too, the microtitre plates are preferred to have 96, 384 or 1536 wells. In another preferred embodiment of the method of the invention, the cellular DNA in step (c) is separated by silica particles.

Within the meaning of this invention, the term “separation of the cellular DNA by means of silica particles” means that the cellular DNA (i.e. the plasmid DNA or the chromosomal DNA in another embodiment) is bound to these particles and separated from the cell debris. In principle, this separation step therefore is a purification step. The silica particles can be removed easily by centrifugation from cell debris.

In a particularly preferred embodiment of the method of the invention, the silica particles are magnetic silica particles.

Thus, the embodiment is particularly preferred as the magnetic particles can easily be removed from the cell debris and other supernatant by using a magnet. Corresponding methods are described, for example, in U.S. Pat. No. 6,027,945 and WO 98/31840.

In a preferred embodiment of the method of the invention, the separation of the endotoxins in step (d) is carried out by precipitation with SDS/isopropanol.

A suitable composition is 2.5% SDS in isopropanol.

In a particularly preferred embodiment of the method of the invention, the DNA bound to silica particles is further purified by washing with acetone.

In another preferred embodiment of the method of the invention, the endotoxins in step (d) are separated by means of endotoxin-binding particles which are preferred to be magnetic endotoxin-binding particles.

The endotoxin-binding particles can preferably be provided as magnetic particles.

In another preferred embodiment of the method of the invention, the transfection of cells in step (e) is mediated by calcium phosphate, electroporation or lipofection.

In another preferred embodiment of the method of the invention, the transfection of cells in step (s) is mediated by calcium phosphate or lipofection. Mediation of the lipofection can be effected by lipids, liposomes or lipid combinations. Examples thereof are Effectene (Qiagen; Hilden), Fugene (Roche; Basle), Metafectene (Biontex), lipofectamins or Lipfectamine 2000, Lipofectin, Oligofectamine (Invitrogen; Karlsruhe).

Metafectene, Oligofectamine or calcium phosphate are particularly suitable for the transfection of RNAi oligonucleotides.

Corresponding methods are known in the state of the art and are described, for instance, in “Transfection Technologies” (Methods Mol. Biol. 2000; 130: 91-102) or Current Protocols (Ausubel et al., 2002; 9.1).

In an additional preferred embodiment of the method of the invention, the transfection is carried out using DNA-binding magnetic biocompatible micro-particles.

The term “biocompatible micro-particle” means micro-particles that are biologically inert or that can be metabolised in a cell.

In this preferred embodiment, modified micro-particles can already be used in the step of DNA preparation, wherein said micro-particles can then be used directly for transfection. The method, which is hereinafter called magneto-transfection, is based on the following parameters:

The DNA suitable for transfection is bound to biocompatible magnetic micro-particles. The micro-particles with the DNA bound thereto are applied to the cell cultures. By application of a magnetic field, the DNA micro-particle complexes are concentrated on the cell surface and taken up into the cell by endocytotoxic processes. Alternatively, the DNA micro-particles can be inserted into the cell/nucleus by increase of the magnetic field. Such a method of magneto-transfection is known in the state of the art and described, for instance, in PCT/EP01/07261. The effectiveness of said method can still be improved by using lipophilic substances that enhance the uptake, e.g. by lipofectamin.

The magnetic concentration of the complexes or the insertion of the DNA micro-particles in the cell/nucleus on the cell surface leads to an increased transfection efficiency. In this way, the amount of sample DNA can be reduced and, with regard to the amount of samples used in a high through-put system, the costs can be reduced significantly. Furthermore, by using said micro-particles, the transfection process can be carried out on a robot system which has similar specifications as the robot system used for DNA preparation. In addition, the process steps can be reduced further and the overall process can be sped up.

This preferred embodiment provides a high through-put transfection system with which a daily through-put rate of up to 40,000 samples can be achieved in a particularly cost- and money-saving manner.

In another preferred embodiment of the method of the invention, the third robot is characterised by at least one and preferably all of the following features: (a) a conveyor road transport system combined with grip arms for microtitre plates for reloading the products and for transferring the microtitre plates between the product processing stations, (b) product processing stations integrated into the transport system, particularly pipetting stations, shakers and incubation places and an incubator for culturing the transfectants, (c) a sensor technology for the detection of product positions as well as for the detection of errors, (d) sterile overpressure ventilation to prevent contaminations of the cell cultures, (e) a software for the interlaced handling of several processes which are in the machine for a continual production process and (f) a computer-based bar code identification and tracking system, preferably with an internal product tracking containing a time stamp function for the interlacing of time-critical sub-processes.

In another preferred embodiment of the method of the invention, the forth robot is characterised by at least one and preferably all of the following features: (a) a system for determining the fluorescence, luminescence or colour reactions from cell culture assays, (b) a pipetting station with a grip arm for microtitre plates for transferring the microtitre plates from the incubator to and between the product processing stations, (c) processing places for adding and withdrawing cell culture media or reagents and incubation in the incubator and (d) computer-based bar code identification and tracking system.

The system is preferably an ELISA reader or a microtitre plate imaging system. It is moreover preferred that the system is suitable for determining the cell morphology. As is the case with the other robots, it is preferred that the microtitre plate has 96 or 384 wells. Apart from processing places for adding and withdrawing cell culture media, etc., the robot may have two other product processing stations such as, e.g. shakers, incubation places.

In an additional preferred embodiment of the method of the invention, the forth robot is characterised by at least one and preferably all of the following features: (a) a digital image processing system and image acquisition system for determining the cell morphology, luminescence and/or fluorescence, (b) a pipetting station with grip arm for microtitre plates for transferring the microtitre plates from the incubator to and between the product processing stations, (c) processing places for adding and withdrawing cell culture media or reagents and incubation in the incubator and (d) a computer-based bar code identification and tracking system.

The term “image processing system” means a system that can detect and analyse automatically differences in the luminescence or fluorescence properties and the morphology of the cells to be examined. Preferably, the data processing of such a system is based on neuronal networks or other corresponding digital image-analytical algorithms of the state of the art.

The term “image acquisition system” is an automated microscoping station which can generate images of the cells to be examined using camera or scanning systems.

In this case, both the image processing and the acquisition system are suitable for a high through-put process.

In still another preferred embodiment of the method of the invention, the automated screening is a functional screening.

Within the meaning of this invention, the term “functional screening” means that the nucleic acid such as DNA or the (poly)peptide encoded thereby is tested for a function. An RNA can be tested for a ribosyme property, an anti-sense property or the binding property within the meaning of an aptamer. Mostly however, the (poly)peptide encoded is tested for a desired property.

An RNAi oligonucleotide (double-stranded RNA) (Elbashir et al., 2002) can be tested for its property to reduce or block the expression of genes.

In a particularly preferred embodiment of the method of the invention, the functional screening is a screening for an enzymatic, pharmacological or therapeutic property.

Said property is usually tested with the (poly)peptide. The property, for instance, to induce apoptosis in the cell can be determined by means of the cell morphology or cell assays such as the CDD+ assay (Roche Diagnostics; Basle/Switzerland) or by caspase activation.

In another preferred embodiment, the functional screening is a screening for the function of secreted proteins. In this case, the proteins encoded by the transfected cDNA are secreted into the cell supernatant. Said supernatant is transferred to target cells and the function of the protein secreted is determined by its effect on the target cell. Alternatively, the cell transfected with the cDNA can be contacted with the target cell and the function of the protein expressed (e.g. on the cell surface) can be determined by its effect on the target cell.

In another particularly preferred embodiment of the method of the invention, the functional screening is a screening for activation or suppression of a reporter system.

Suitable reporter systems are known in the state of the art and comprise reporter gene assays (e.g. for transcriptional activation of indicator proteins, enzymatic activation/deactivation of indicator proteins). Examples thereof are the green fluorescent protein (GFP), luciferase (Firefly) from the field of fluorescence-based reporter systems.

In other preferred embodiments, the screening is a screening for modified cell morphology, cell death or proliferation.

In a preferred embodiment of the method of the invention, 2, 3 or all 4 robots are arranged in a conveyor road.

In this preferred embodiment, at least 2, i.e. 3 or all 4, individual processing stations/robots for colony picking, DNA preparation, DNA transfection and reading-out of the functional screening assay are additionally connected or combined by conveyor road systems. In this way, intermediary steps between the individual processes, which have so far been necessary, are avoided and the sample through-put rate is increased further.

By using conveyer belt transport systems in combination with overhead manipulators by a corresponding interlacing of the process steps, a serial production process is arrived at which, in contrast to classical pipetting stations, has no limitation with respect to the production volume. If alternatively 96-well or 384-well plates are used, flexibility is even more increased.

In a further preferred embodiment of the process according to the invention, a DNA, (poly)peptide or a transfectant containing these which has been identified in a screening process, is purified or isolated.

For the further processing of the DNA/RNAi oligonucleotides/(poly)peptides which were tested positively in the screening process, it is desirable that the substances or the corresponding tranfectant is purified to a no longer contaminated and thus pure form. This is particularly easy with the process of the invention, as e.g. the positively tested substance is directly available by referring back to the master plate. The further purification steps for the substances or the corresponding transfectants can be carried out according to the conventional processes.

In another preferred embodiment, the present invention also relates to a process for improvement of the binding properties of the (poly)peptide encoded by the DNA identified or isolated in the screening process of the invention, comprising the steps of (a) identification of the binding sites of the (poly)peptide or its binding partner by site specific mutagenesis or chimeric protein studies; (b) molecular modelling of the binding site of both the (poly)peptide and the binding partner; and (c) modification of the (poly)peptide in order to improve the binding specificity or the affinity of the binding.

The (poly)peptide can be modified so as to increase the binding affinity or effectiveness and specificity. If e.g. electrostatic interactions between a certain residue of the (poly)peptide in question and a region of the (poly)peptide exists, the total charge of this region can be changed in order to increase the existing interation in this manner.

Computer programs can be useful for identifying binding sites. Thus, suitable computer programs can be used for identifying interactive sites of an alleged inhibitor and the polypeptide by computer-based screening for complementary structural motifs (Fassina, Immunomethods 5 (1994), 114-120). Further suitable computer systems for the computer-based design of proteins and peptides are described in the state of the art, e.g. in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. Modifications of the (poly)peptide can be achieved by e.g. peptidomimetics. Other inhibitors can also be identified by means of synthesis of combinatorial peptidomimetic libraries by successive chemical modification and testing of the compositions which have been obtained. Processes for the production and use of combined peptidomimetic libraries are described in the state of the art, e.g. in Ostresh, Methods in Enzymology 267 (1996), 220-234 and Dorner, Bioorg. Med. Chem. 4 (1996), 709-715. Moreover, the three-dimensional and/or crystallographic structure of the activators of the expression of the (poly)peptide of the invention can be used for the design of peptidomimetic activators, e.g. in connection with the (poly)peptide identified according to the invention (Rose, Biochemistry 35 (1996), 12933-12944, Rutenber, Bioorg. Med. Chem. 4 (1996), 1545-1558).

In a particularly preferred embodiment of the process of the invention, the modification in step (c) is a reproduction of the (poly)peptide by petidomimetics.

In an additional preferred embodiment of the process of the invention, the (poly)peptide as leading structure is further modified in order to obtain (i) a modified site of action, a modified spectrum of activity, a modified organ specificity and/or (ii) an improved activity and/or (iii) a reduced toxicity (an improved therapeutic index) and/or (iv) reduced side effect and/or (v) a delayed on-set of the therapeutic action, of the duration of the therapeutic effect and/or (vi) modified pharmacokinetic parameters (resorption, distribution, metabolism or excretion) and/or (vii) modified physicochemical parameters (solubility, hygroscopic properties, colour, taste, odour, stability, state) and/or (viii), improved general specificity, organ/tissue specificity and/or (ix) optimised application form and route by (i) esterification of carboxylic groups or (ii) esterification of hydroxyl groups with carboxylic acids or (iii) esterification of hydroxyl groups to form e.g. phosphates, pyrophosphates or sulfates or amber acid semi-esters or (iv) formation of pharmaceutically acceptable salts or (v) the formation of pharmaceutically acceptable complexes or (vi) the synthesis of pharmaceutically active polymers or (vii) the introduction of hydrophilic moieties or (viii) the introduction/exchange of substituents in aromates or side chains, change of the substituent pattern or (ix) modification by introduction of isosteric or bioisosteric moieties or (x) the synthesis of homologous compounds or (xi) introduction of branched side chains or (xii) conversion of alkyl substituents to form cyclic analogues or (xiii) derivatisation of hydroxyl groups to form ketals or acetals or (xiv) N-acetylation to form amides, phenylic carbamates or (xv) synthesis of Mannich bases, imines or (xvi) transformation of ketones or aldehydes to Schiff's bases, oximes, acetals, ketals, enolic esters, oxazolidines, thiozolidines or combinations thereof.

The different above-mentioned steps are generally known in the art. They comprise or are based on quantitative structure-effect-relationships (QSAR) analyses (Kubinyi, “Hausch-Analysis and Related Approaches”, VCH Verlag, Weinheim, 1992), combined biochemistry, classical chemistry and others (cf. e.g. Holzgrabe and Bechtold, Deutsche Apotheker Zeitung 140(8), 813-823, 2000).

Moreover, the present invention relates to a process for the production of a pharmaceutical composition comprising the steps of the process of the invention and furthermore the formulation of the substance obtained with a pharmaceutically acceptable carrier or diluent.

The pharmaceutical composition can be produced in a conventional manner.

Examples of suitable pharmaceutically acceptable carriers and/or diluents are known to the person skilled in the art and comprise e.g. phosphate buffered physiological salines, water, emulsions, such as e.g. oil/water emulsions, different kinds of wetting agents or detergents, sterile solutions, etc. Pharmaceutical compositions comprising such carriers can be formulated by means of known conventional processes. These pharmaceutical compositions can be administered to an individual in a suitable dose. The administration can be effected orally or parenteraly, e.g. intravenously, intraperitoneally, subcutaneously, intramuscularly, locally, intranasally, intrabronchially or intradermally or by means of a catheter somewhere in an artery. The dosage form is chosen by the physician in charge according to the clinical factors. It is known to the person skilled in the art that the dosage form depends on several factors such as e.g. the body size or the weight, the body surface, the age, the sex or the general health of the patient but also on the substance to be administered in particular, the duration and form of the administration and on other pharmaceutical preparations which are possibly administered at the same time. A typical dose can e.g. be in a range from 0.001 to 1,000 μg, with doses below or above this exemplary range being possible, in particular when considering the above-identified factors. In general, the dose should range from 1 μg and 10 mg units per day if the composition of the invention is administered regularly. If the composition is administered intravenously, which is not recommended as being preferred in order to minimize the danger of anaphylactic reactions, the dose should range from 1 μg and 10 mg units per kilogram body weight per minute.

The composition of the invention can be administered locally or systemically. Preparations for a parenteral administration comprise sterile aqueous or non-aqueous solutions, suspensions and emulsions. Examples of non-aqueous solvents are propylene glycol, polyethylene glycol, plant oils such as e.g. olive oil and organic ester compositions such as e.g. ethyloleate which are suitable for injections. Aqueous carriers comprise water, alcoholic-aqueous solutions, emulsions, suspensions, saline solutions and buffered media. Parenteral carriers comprise sodium chloride solutions, Ringer's dextrose, dextrose and sodium chloride, Ringer's lactate and bound oils. Intravenous carrier comprise e.g. fluid, nutrient and electrolyte supplements (such as e.g. those based on Ringer's dextrose). The composition according to the invention can moreover comprise preserving agents and other additives such as e.g. antimicrobial compounds, antioxidants, complex former and inert gasses. Moreover, dependent on the intended use, compounds such as e.g. interleukins, growth factors, differentiation factors, interferons, chemotactic proteins or an unspecific immunomodulatory agent can be contained.

In general, the complete process on which the invention is based can e.g. be presented as follows:

1. Picking the Bacterial Colonies and Replication (Robot 1)

cDNA banks are plated on agar plates, the individual colonies are picked and transferred to microtitre plates where the bacteria are cultivated for propagation. In a second step, several growth plates are inoculated from these master plates and are cultivated for propagation to generate sufficient bacteria for the isolation of the DNA (replication).

2. DNA Preparation (Robot 2)

The growth plates with the bacterial suspension are centrifuged and the supernatant is sucked off. Subsequently, the pellets are resuspended in a buffer containing RNAse (P1), an alkaline lysis buffer (P2) is added and is then neutralised (P3).

These steps are carried out on an orbital shaker to which a multi-channel dispenser is fixed.

After a short incubation, the plates are centrifuged and the supernatant is transferred to a support plate. Subsequently, P4 is dispended in order to bind bacterial endotoxins, is recentrifuged after an incubation and the supernatant is transferred to a second support plate. Silica is dispensed to this supernatant in order to bind the DNA. A centrifugation is carried out, the supernatant is removed and the pellet is washed with acetone. After having carried out another centrifugation, the acetone supernatant is sucked off, the silica pellet is resuspended with hot water with a temperature of 60° C. (removal of the DNA), centrifuged and the DNA-solution is transferred to the final plates. (Buffer 1: Tris EDTA with RNAse, P2: NaOH/SDS, P3: potassium acetate buffer, P4: SDS in isopropanol).

3. DNA Transfection (robot 3) A defined amount of the DNA solution from the DNA plates produced by robot 2 is pipetted in support plates and a control plasmid (β-Gal), calcium chloride, HBS are added. After an incubation for complex formation chloroquine is dispensed to the preparation and after mixing, a defined amount of the preparation is pipetted onto the cell culture. After 4 to 5 hours, the medium is changed.

4. Functional Screening Assay (Robot 4)

After 24 to 48 hours, a substrate is added to the cell culture plates which causes a change in colour in apoptotic cells. This change in colour is evaluated in the ELISA reader and the cells are discarded.

2) DNA Preparation and Transfection Method by Using Magnetic Micro-Particles:

After their growth, bacteria are centrifuged in growth plates and are treated with an RNAse buffer. The bacteria are resuspended on an orbital shaker. Subsequently, a lysis and a neutralising buffer are added. By adding a first kind of magnetic micro-particle, cell debris and proteins are bound. The magnetic micro-particles are separated on a magnetic plate and the supernatant is transferred to a support plate. Afterwards, optionally, a second kind of magnetic micro-particle is added which bind to bacterial endotoxins. These are also separated magnetically and the supernatant is transferred into a second support plate. These steps can be combined by adding a mixture of both kinds of micro-particles.

Alternatively, endotoxin precipitation reagents can be used which are removed after the precipitation of the endotoxins in the first micro-particle separation step.

In a last step, magnetic micro-particles are added which bind to the DNA. The DNA can either be eluted from these magnetic micro-particles and used for transfection or, if the micro-particles are formulated accordingly, it can be used directly for transfections.

In a preferred embodiment, the DNA micro-particle complexes produced during the DNA isolation can be used directly for transfections.

The example illustrates the invention.

### EXAMPLE 1

### Carrying Out the Screening Method for the Determination of the Function of Genes or Gene Products

1. Colony Picking and Replication

The bacteria containing DNA were plated in such a way with a selection antibiotic on agar plates that as high an amount as possible of single clones was evenly distributed on the plates. After an overnight incubation at 37° C., the colonies were picked by a robot and were transferred into microtitre plates with 384 wells (MTP), in which 60 μl LB medium with a selection antibiotic was present. These plates were incubated overnight at 37° C. and, on the following day, were coated with a mixture of LB medium and glycerine so that the final concentration of glycerine amounted to 15%. Subsequently, the plates (hereinafter referred to as master MTP) were stored at −80° C.

For further use, the master MTPs were thawed and replicated with a replication tool on a first robot in 4×“Deepwell” MTP with 96 wells. 1.5 ml LB medium with a selection antibiotic was plated into each of these 96-well MTPs. After inoculation, the plates were incubated overnight in a shaking container, the shaking speed amounting to 280 rpm.

2. DNA Preparation

The MTPs with 96 wells were centrifuged at 3,000 g for 5 minutes and the supernatant was removed. 170 μl P1 (50 mM Tris pH 8.0; 10 mM EDTA pH 8.0; 100 μg/ml RNAse A (Qiagen) were added on a shaking station with dispenser, shaken at 1,000 rpm for 5 minutes, 170 μl P2 (200 mM NaOH, 1% SDS) were added, shaken for 10 s at 300 rpm and incubated at room temperature for 5 minutes. Subsequently, 170 μl P3 (3 M KAc, pH 5.5) were added and shaken for 30 seconds at 1,000 rpm. After 5 minutes of incubation at 4° C., the MTPs were centrifuged for 5 minutes at 3,500 g. The supernatant was removed and was transferred to a support MTP. 120 μl P4 (2.5% SDS (Roth) in isopropanol) were added to the supernatant and were incubated for 20 minutes at 4° C. Subsequently, a centrifugation was carried out for 10 minutes at 3,500 g and the supernatant was transferred onto a support plate. 120 μl silica (50 mg/ml SiO₂ (12.5 g per 250 ml water)) were added and incubated for 5 minutes at room temperature. In this case, the silica suspension was prepared as follows: 12.5 g silica per 250 ml water was stirred for 30 minutes, the supernatant (contains silica powder) was sedimented; removed; 150 μl concentrated HCl was added, filled up with H₂O to 250 ml (graduated cylinder) and autoclaved. Subsequently, a centrifugation was carried out for 5 minutes at 2,000 g and the supernatant was discarded. 400 μl acetone were added, shaken for 1 minute at 1,000 rpm and subsequently centrifuged for 5 minutes at 2,000 g. Then, the supernatant was sucked off and the plates with the silica pellets were dries for 20 minutes on a heating plate at 70° C. Subsequently, 140 μl bidistilled water was added at a temperature of 65° C., was shaken for 5 minutes at 800 rpm, centrifuged for 5 minutes at 3,000 g and the supernatant was stored with the DNA in a 96-well polystyrene MTP.

3. Transfection

On the day prior to the transfection, the cells to be transfected were plated with a cell density of approximately 8,000 cells/well in a 96 well cell culture plate. 5 β-Gal plasmid (c=100 ng/l) were dispensed in a support MTP and subsequently 20 μl of the DNA solution (c=100 ng/l) were added. Subsequently 20 μl (0.25 M CaCl₂) were added, briefly shaken and subsequently 25 μl L2 (2×HBS) were added. After an incubation for 20 minutes at room temperature, 15 μL3 (2 mM chlorochin-solution) were added and briefly shaken. 91 μl of this mixture were placed on the cells and incubated 5 to 6 at 37° C. Subsequently, a medium change (DMEM/10% FCS) was carried out. After an incubation overnight the medium (DMEM/10% FCS) was changed again.

4. Functional Reading Out

30 μl CPRG solution 2.31 ml, 0.1 M sodium phosphate solution, 30 μl 100×MgCl₂, 660 μl CPRG solution were added to the transfected cell to each well of the cell culture plate and incubated for 1 to 3 hours. Subsequently, the plates were measured in an ELISA reader (absorption measurement at 570 nm).

### EXAMPLE 2

### Functional Screening for Secreted Proteins

COS-7 cells are seeded with a cell density of approximately 5,000 cells/well in 10% DMEM and incubated for 24 hours at 37° C. in an incubator. The cDNA is introduced into the cells by lipofection with Metafectene (Biontex, Munich) and incubated for 3 hours at 37° C. in the incubator. After complete removal of the medium, the endothelial cell growth medium (PromoCell, Heidelberg) is added and the cells are incubated in an incubator for 48 hours at 37° C. Subsequently, the supernatant is removed and is transferred to the endothelial cells (human umbelical vein endothelial cells, HUVECs or microvascular endothelial cells, HMVECs). Beforehand, these HUVEC cells are seeded with a cell density of 2,000 cells/well in endothelial cell growth medium (PromoCell, Heidelberg). After complete removal of the medium, the supernatant of the COS-7 cells is transferred to the endothelial cells. The cells are incubated for 6 days at 37° C. in an incubator and the activities of the secreted proteins are determined by the cytosolic reduction of Alamar Blue (BioSource, Solingen).

If no other indications are given, the individual assay steps are carried out with protocols according to Current Protocols (Ausubel et. al, 2002).

1. A method for screening a collection of nucleic acid molecules for a desired property of the nucleic acid or of a (poly)peptide encoded thereby, comprising the steps of
(a) automated picking of a collection of cells containing the collection of nucleic acid molecules by means of a first robot;
(b) automated lysis of the cells by means of a second robot;
(c) automated separation of the cellular DNA from the cell debris by means of a second robot;
(d) optionally automated separation of endotoxins from the DNA by means of the second robot if the cells are bacteria;
(e) automated transfection of cells with the DNA obtained in step (c) or, if the cells are bacteria, obtained in step (d) by means of a third robot; and
(f) automated screening for the desired property by means of a fourth robot.

2. The method according to claim 1 wherein the collection of nucleic acid molecules is a gene library or a collection of clones.

3. The method according to claim 1 or 2 wherein the nucleic acid molecules are genomic DNA or cDNA molecules or RNAi oligonucleotides.

4. The method according to claim 2 or 3 wherein the gene library is an expression cDNA gene library, preferably a eukaryotic gene library, a human gene library is particularly preferred.

5. The method according to any one of claims 1 to 4 wherein the cells in step (a) and/or step (e) are mammalian cells, insect cells, yeast cells or bacteria.

6. The method of claim 5 wherein the bacteria are Gram-negative bacteria.

7. The method of claim 6 wherein the Gram-negative bacteria belong to the species E. coli.

8. The method of any one of claims 1 to 7 wherein at least one of the steps (a) to (f) is carried out in microtitre plates.

9. The method according to claim 8 wherein all steps (a) to (f) are carried out in microtitre plates.

10. The method according to claim 8 or 9 wherein the microtitre plates have bar codes.

11. The method according to any one of claims 1 to 10 wherein the first robot is characterised by
(a) a digital image processing system for collecting the plated bacteria,
(b) a working station with a grip arm for microtitre plates for transferring the microtitre plates between the processing stations,
(c) a separation module having one or more heads with needles for picking the plated single colonies and for placing them into the microtitre plates,
(d) integrated product processing stations for cleaning the needles between the working steps and replicating the placed single colonies in the microtitre plates and
(e) a computer-based bar code identification and tracking system.

12. The method of any one of claims 1 to 4 wherein the lysis is an alkaline lysis.

13. The method of any one of claims 1 to 12 wherein the second robot is characterised by
(a) a conveyor road transport system combined with grip arms for the microtitre plates for reloading the products and for transferring the microtitre plates between the product processing stations,
(b) product processing stations integrated into the transport system, particularly centrifuges, pipetting automats, shakers and incubation places for incubation at different temperatures,
(c) a sensor technology for the detection of product positions as well as for the detection of errors,
(d) a software for the interlaced handling of several processes which are in the machine for a continual production process and
(e) a computer-based bar code identification and tracking system, preferably with an internal product tracking containing a time stamp function for the interlacing of time-critical sub-processes.

14. The method according to any one of claims 1 to 13 wherein the separation of the cellular DNA in step (c) is carried out with silica particles.

15. The method according to claim 14 wherein the silica particles are magnetic silica particles.

16. The method according to any one of claims 1 to 15 wherein the separation of the endotoxins in step (d) is carried out with endotoxin-binding particles, which are preferably magnetic endotoxin-binding particles.

17. The method according to any one of claims 1 to 15 wherein the separation of the endotoxins in step (d) is carried out by precipitation with SDS/isopropanol.

18. The method according to any one of claims 14 to 16 wherein the DNA bound to silica particles is further purified by washing with acetone.

19. The method according to any one of claims 1 to 18 wherein the transfection of cells in step (e) is mediated by calcium phosphate, electroporation or by lipofactors.

20. The method according to any one of claims 1 to 18 wherein the transfection is carried out by means of DNA-binding magnetic biocompatible micro-particles.

21. The method of any one of claims 1 to 20 wherein the third robot is characterised by
(a) a conveyor road transport system combined with grip arms for microtitre plates for reloading the products and for transferring the microtitre plates between the product processing stations,
(b) product processing stations integrated into the transport system, particularly pipetting stations, shakers and incubation places and an incubator for culturing the transfectants,
(c) a sensor technology for the detection of product positions as well as for the detection of errors,
(d) sterile overpressure ventilation to prevent contaminations of the cell cultures,
(e) a software for the interlaced handling of several processes which are in the machine for a continual production process and
(f) a computer-based bar code identification and tracking system, preferably with an internal product tracking containing a time stamp function for the interlacing of time-critical sub-processes.

22. The method of any one of claims 1 to 21 wherein the fourth robot is characterised by
(a) a system for determining the fluorescence, luminescence or colour reactions from cell culture assays,
(b) a pipetting station with a grip arm for microtitre plates for transferring the microtitre plates from the incubator to and between the product processing stations,
(c) processing places for adding and withdrawing cell culture media or reagents and incubation in the incubator and
(d) computer-based bar code identification and tracking system.

23. The method according to any one of claims 1 to 21 wherein the fourth robot is characterised by
(a) a digital image processing system and image acquisition system for determining the cell morphology, fluorescence and/or luminescence
(b) a pipetting station with grip arm for microtitre plates for transferring the microtitre plates from the incubator to and between the product processing stations,
(c) processing places for adding and withdrawing cell culture media or reagents and incubation in the incubator and
(d) a computer-based bar code identification and tracking system.

24. The method according to any one of claims 1 to 23 wherein the automated screening is a functional screening.

25. The method according to claim 24 wherein the functional screening is a screening for an enzymatic, pharmacological or therapeutic property.

26. The method according to claim 14 or 25 wherein the functional screening is a screening for activation or suppression of a reporter system or wherein the screening is a screening for the function of a secreted protein.

27. The method according to any one of claims 1 to 26 wherein 2, 3 or 4 robots are arranged in a conveyor road.

28. The method according to any one of claims 1 to 27 wherein a DNA, (poly)peptide or a transfectant containing the same, which has been identified in the screening process is purified or isolated.

29. The method according to any one of claims 1 to 28 which moreover comprises the improvement of the binding properties of the (poly)peptide encoded by the DNA identified or isolated in the screening process according to any one of claims 1 to 28, comprising the steps of
(a) identification of the binding sites of the (poly)peptide or its binding partner by site-specific mutagenesis or chimeric protein studies;
(b) molecular modelling of the binding site of the (poly)peptide and of the binding partner; and
(c) modification of the (poly)peptide in order to improve the binding specificity or the affinity of the binding.

30. The method according to claim 29 wherein the modification in step (c) is a reproduction of the (poly)peptide by peptidomimetics.

31. The method according to any one of claims 1 to 28 wherein the (poly)peptide as a leading structure is further modified in order to obtain
(i) a modified site of action, a modified spectrum of activity, a modified organ specificity, and/or
(ii) an improved activity, and/or
(iii) a decreased toxicity (an improved therapeutic index), and/or
(iv) decreased side effects, and/or
(v) a delayed onset of the therapeutic action, of the duration of the therapeutic effect and/or
(vi) modified pharmacokinetic parameters (resorption, distribution, metabolism or exretion), and/or
(vii) modified physico-chemical parameters (solubility, hygroscopic properties, colour, taste, odour, stability, state), and/or
(viii) improved general specificity, organ/tissue specificity, and/or
(ix) optimised application form and route by
(i) esterification of carboxyl groups, or
(ii) esterification of hydroxyl groups with carboxylic acids, or
(iii) esterification of hydroxyl groups to e.g. phosphates, pyrophosphates or sulfates or succinic acid semiesters, or
(iv) formation of pharmaceutically acceptable salts, or
(v) formation of pharmaceutically acceptable complexes, or
(vi) synthesis of pharmacologically active polymers, or
(vii) introduction of hydrophilic moieties, or
(viii) introduction/exchange of substituents in aromates or side chains, change of the substituent pattern, or
(ix) modification by introduction of isosteric or bioisosteric moieties, or
(x) synthesis of homologous compounds, or
(xi) introduction of branched side chains, or
(xii) conversion of alkyl substituents to cyclic analogues, or
(xiii) derivatisation of hydroxyl groups to ketals or acetals, or
(xiv) N-acetylation to amides, phenylcarbamates, or
(xv) synthesis of Mannich bases, imines, or
(xvi) transformation of ketones or aldehydes to Schiff's bases, oximes, acetals, ketals, enolic esters, oxazolidines, thiozolidines or combinations thereof.

32. The method for the manufacture of a pharmaceutical composition comprising the steps of the method according to any one of claims 23 to 31 and, moreover, formulating of the substance obtained with a pharmaceutical acceptable carrier or diluent.
